(19) United States
(12) Patent Application Publication
(10) Pub. No.: US 2024/0052040 A1
(43) Pub. Date: Feb. 15, 2024

(54) Sialylated Glycoproteins

(71) Applicant: Momenta Pharmaceuticals, Inc., Titusville, NJ (US)

(72) Inventors: Birgit C. Schultes, Arlington, MA (US); Chia Lin Chu, Somerville, MA (US); Laura Rutitzky, Somerville, MA (US); Lynn Zhang, Acton, MA (US); Leona E. Ling, Winchester, MA (US)

(21) Appl. No.: 18/136,803

(22) Filed: Apr. 19, 2023

Related U.S. Application Data
(63) Continuation of application No. 16/688,772, filed on Nov. 19, 2019, now abandoned.
Application No. 16/688,772 is a continuation of application No. 15/985,288, filed on May 21, 2018, now Pat. No. 11,661,456.
Application No. 15/985,288 is a continuation of application No. 15/028,917, filed on Apr. 12, 2016, now abandoned.
(60) Provisional application No. 61/891,778, filed on Oct. 16, 2013.

(51) Int. Cl. C07K 16/28; C07K 16/00; C07K 16/06; A61K 39/395; A61P 7/00

(52) U.S. Cl. CPC C07K 16/2848; C07K 16/00; C07K 16/06; A61K 39/39516; A61P 7/00; A61K 2039/505

(57) ABSTRACT

Pharmaceutical preparations containing polypeptides having particular sialylation patterns, and methods for the treatment of immune-related thrombocytopenia with such preparations, are described.

### CROSS REFERENCE TO RELATED APPLICATIONS

This application is a continuation of U.S. patent application Ser. No. 16/688,772, filed on Nov. 19, 2019, which is a continuation of U.S. patent application Ser. No. 15/985,288, filed on May 21, 2018, which is a continuation of U.S. patent application Ser. No. 15/028,917, filed on Apr. 12, 2016 (now abandoned), which is national stage application under 35 U.S.C. § 371 of International Application Number PCT/US2014/060363, filed on Oct. 14, 2014, which claims the benefit of U.S. Provisional Application No. 61/891,778, filed Oct. 16, 2013, which are hereby incorporated by reference in their entirety.

### SEQUENCE LISTING

This application contains a Sequence Listing that has been submitted electronically as an XML file named “14131-0144004_SL_ST26.XML.” The XML file, created on Apr. 20, 2023, is 4,779 bytes in size. The material in the XML file is hereby incorporated by reference in its entirety.

### BACKGROUND

Therapeutic glycoproteins are an important class of therapeutic biotechnology products, and therapeutic Fc containing glycoproteins, such as IVIg, Fc-receptor fusions, and antibodies (including murine, chimeric, humanized, and human antibodies and fragments thereof) account for the majority of therapeutic biologic products.

### SUMMARY OF THE INVENTION

The invention encompasses, in part, the discovery that Fc-containing polypeptides that include branched glycans and that are di-sialylated on the branched glycan (e.g., on an α 1,3 and/or α 1,6 arm in the Fc region's N-linked glycosylation site), with, e.g., a NeuAc-α 2,6-Gal terminal linkage, exhibit improved biological activity, e.g., relative to a reference glycoprotein, e.g., in the treatment of hematological disease, e.g., immune-related thrombocytopenia (ITP). The present disclosure provides, in part, methods for treating hematological disease, e.g., immune-related thrombocytopenia and related diseases by administering compositions containing such Fc-containing polypeptides as well as methods for evaluating, identifying, and/or producing (e.g., manufacturing) such polypeptides.

In one aspect, the invention features a pharmaceutical preparation formulated for subcutaneous administration (e.g., at a concentration of 50-250 mg/mL, e.g., 50-100 mg/mL, 75-125 mg/mL, 100-150 mg/mL, 125-175 mg/mL, 150-200 mg/mL, 175-225 mg/mL, 200-250 mg/mL). This preparation includes polypeptides having an Fc region, wherein at least 50% (e.g., 60%, 70%, 80%, 82%, 85%, 87%, 90%, 92%, 94%, 95%, 97%, 98% up to and including 100%) of branched glycans on the Fc region are di-sialylated by way of NeuAc-α 2,6-Gal terminal linkages. In some embodiments, less than 50% (e.g., less than 40%, 30%, 20%, 15%, 10%, 5%, 4%, 3%, 2%, 1%) of branched glycans on the Fc region are mono-sialylated (e.g., on the α 1,3 arm or the α 1,6 arm) by way of a NeuAc-α 2,6-Gal terminal linkage.

In another aspect, the invention features a pharmaceutical preparation including polypeptides having an Fc region, wherein at least 50% (e.g., 60%, 70%, 80%, 82%, 85%, 87%, 90%, 92%, 94%, 95%, 97%, 98% up to and including 100%) of branched glycans on the Fc region are di-sialylated by way of NeuAc-α 2,6-Gal terminal linkages and less than 50% (e.g., less than 40%, 30%, 20%, 10%, 15%, 5%, 4%, 3%, 2%, 1%) of branched glycans on the Fc region are mono-sialylated on the α 1,3 arm by way of a NeuAc-α 2,6-Gal terminal linkage.

In another aspect, the invention features a pharmaceutical preparation comprising polypeptides having an Fc region, wherein at least 50% (e.g., 60%, 70%, 80%, 82%, 85%, 87%, 90%, 92%, 94%, 95%, 97%, 98% up to and including 100%) of branched glycans on the Fc region are di-sialylated by way of NeuAc-α 2,6-Gal terminal linkages and less than 50% (e.g., less than 40%, 30%, 20%, 10%, 5%, 4%, 3%, 2%, 1%) of branched glycans on the Fc region are mono-sialylated on the a 1,6 arm by way of a NeuAc-α 2,6-Gal terminal linkage.

In another aspect, the invention features a pharmaceutical preparation comprising polypeptides having an Fc region, wherein at least 85% of branched glycans on the Fc region are di-sialylated by way of NeuAc-α 2,6-Gal terminal linkages.

In some embodiments of any of the foregoing preparations, the polypeptides consist essentially of an Fc region. In other embodiments of any of the foregoing preparations, the polypeptides further include a Fab region, a heterologous polypeptide sequence such as a biological receptor sequence (e.g., the polypeptides are Fc-receptor fusion proteins), or a heterologous non-polypeptide moiety.

In certain embodiments, at least 10% (e.g., 20%, 30%, 40%, 50%, 60% 70% or more) of branched glycans on the Fab region or heterologous polypeptide sequence of the polypeptides are mono-sialylated or di-sialylated. In other embodiments, less than 80% (e.g., 70%, 60, 50%, 40%, 30%, 20%, 10%, 5% or less) of branched glycans on the Fab region or heterologous polypeptide sequence of the polypeptides are mono-sialylated or di-sialylated.

In some embodiments of any of the foregoing preparations, the polypeptides are recombinant polypeptides. In other embodiments of any of the foregoing preparations, the polypeptides are derived from plasma, e.g., human plasma. In certain embodiments, the polypeptides are IgG polypeptides (e.g., IgG1, IgG2, IgG3 or IgG4) or the polypeptides consist essentially of an Fc region derived from IgG polypeptides.

In another aspect, the invention features a method of increasing reticulated platelets in a subject in need thereof, comprising administering to the subject any one of the foregoing preparations.

In another aspect, the invention features a method of producing new platelets in a subject in need thereof, comprising administering to the subject any one of the foregoing preparations.

In another aspect, the invention features a method of increasing reticulated platelets or producing new platelets in a subject in need thereof, comprising administering to the subject a pharmaceutical preparation comprising polypeptides comprising an Fc region, wherein at least 85% of branched glycans on the Fc region are di-sialylated by way of NeuAc-α 2,6-Gal terminal linkages.

In some embodiments of any of the foregoing methods, the subject is not being treated with thrombopoietin or a thrombopoietin receptor agonist (e.g., romiplostim, eltrombopag). In some embodiments of any of the foregoing methods, the subject has failed treatment with thrombopoietin or a thrombopoietin receptor agonist (e.g., romiplostim, eltrombopag). In other embodiments of any of the foregoing methods, the subject has a hematological disease such as immune-related thrombocytopenia. In certain embodiments of any of the foregoing methods, the method further includes, after the administering step, the step of determining the total platelet count and/or the reticulated platelet count in the subject, e.g., wherein the total platelet count and/or the reticulated platelet count increases as a result of the administering step. In some embodiments, the method further includes after the determining step, the step of adjusting the dose of the administered pharmaceutical preparation.

### DESCRIPTION OF THE FIGURES

FIG. 1 is a schematic illustration of a common core pentasaccharide (Man)₃(GlcNAc)(GlcNAc) of N-glycans.

FIG. 2 is a schematic illustration of an IgG antibody molecule.

FIG. 3A depicts an exemplary ST6 sialyltransferase amino acid sequence (SEQ ID NO:1). FIG. 3B depicts an exemplary ST6 sialyltransferase amino acid sequence (SEQ ID NO:2). FIG. 3C depicts an exemplary ST6 sialyltransferase amino acid sequence (SEQ ID NO:3).

FIG. 4 is a schematic illustration of a reaction scheme for ST6 sialyltransferase (fucose: triangles, N-acetylglucosamine: squares, mannose: dark circles, galactose: light circles, sialic acid: diamonds).

FIG. 5 is a graphic representation of relative abundance of glycans at various times during a sialylation reaction with ST6 sialyltransferase.

### DETAILED DESCRIPTION OF THE INVENTION

Antibodies are glycosylated at conserved positions in the constant regions of their heavy chain. For example, IgG antibodies have a single N-linked glycosylation site at Asn297 of the CH2 domain. Each antibody isotype has a distinct variety of N-linked carbohydrate structures in the constant regions. For human IgG, the core oligosaccharide normally consists of GlcNAc₂Man₃GlcNAc, with differing numbers of outer residues. Variation among individual IgG's can occur via attachment of galactose and/or galactose-sialic acid at one or both terminal GlcNAc or via attachment of a third GlcNAc arm (bisecting GlcNAc).

The present disclosure encompasses, in part, pharmaceutical preparations including polypeptides having an Fc region having particular levels of branched glycans that are sialylated on both of the branched glycans in the Fc region (e.g., with a NeuAc-α 2,6-Gal terminal linkage). The levels can be measured on an individual Fc region (e.g., the number of branched glycans that are sialylated on an α1,3 arm, an α1,6 arm, or both, of the branched glycans in the Fc region), or on the overall composition of a preparation of polypeptides (e.g., the number or percentage of branched glycans that are sialylated on an α1,3 arm, an α1,6 arm, or both, of the branched glycans in the Fc region in a preparation of polypeptides).

The inventors have discovered that Fc-region containing polypeptides having branched glycans that are preferentially di-sialylated (e.g., with NeuAc-α 2,6-Gal terminal linkages) exhibit improved biological activity, e.g., relative to a reference glycoprotein, and are useful in the treatment of immune-related thrombocytopenia and related diseases.

Preparations useful herein can be obtained from any source. In some instances, providing or obtaining a preparation (e.g., such as a biologic drug substance or a precursor thereof), e.g., that is or includes a polypeptide, can include providing a host cell, e.g., a mammalian host cell (e.g., a CHO cell) that is genetically engineered to express a polypeptide (e.g., a genetically engineered cell); culturing the host cell under conditions suitable to express the polypeptide (e.g., mRNA and/or protein); and, optionally, purifying the expressed polypeptide, e.g., in the form of a recombinant fusion protein) from the cultured cell, thereby producing a preparation.

### Definitions

As used herein, “acquire or acquiring (e.g., acquiring information)” means obtaining possession of a physical entity, or a value, e.g., a numerical value, by “directly acquiring” or “indirectly acquiring” the physical entity or value. “Directly acquiring” means performing a process (e.g., performing an assay or test on a sample or “analyzing a sample” as that term is defined herein) to obtain the physical entity or value. “Indirectly acquiring” refers to receiving the physical entity or value from another party or source (e.g., a third party laboratory that directly acquired the physical entity or value). “Directly acquiring” a physical entity includes performing a process, e.g., analyzing a sample, that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. “Directly acquiring” a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent. Exemplary analytical methods are shown in Table 1.

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

As used herein, a “batch” of a preparation refers to a single production run. Evaluation of different batches thus means evaluation of different production runs or batches. As used herein “sample(s)” refer to separately procured samples. For example, evaluation of separate samples could mean evaluation of different containers or vials of the same batch or from different batches. A batch can include a drug substance batch or a drug product batch.

As used herein, the term “constant region” refers to a polypeptide that corresponds to, or is derived from, one or more constant region immunoglobulin domains of an antibody. A constant region can include any or all of the following immunoglobulin domains: a C_{H}1 domain, a hinge region, a C_{H}2 domain, a C_{H}3 domain (derived from an IgA, IgD, IgG, IgE, or IgM), and a C_{H}4 domain (derived from an IgE or IgM).

As used herein, “evaluating,” e.g., in the evaluation/evaluating, identifying, and/or producing aspects disclosed herein, means reviewing, considering, determining, assessing, analyzing, measuring, and/or detecting the presence, absence, level, and/or ratio of one or more parameters in a preparation to provide information pertaining to the one or more parameters. In some instances, evaluating can include performing a process that involves a physical change in a sample or another substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. “Evaluating” can include performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, the term “Fc region” refers to a dimer of two “Fc polypeptides,” each “Fc polypeptide” including the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or the entire flexible hinge N-terminal to these domains. For IgG, “Fc polypeptide” comprises immunoglobulin domains Cgamma2 (Cγ2) and Cgamma3 (Cγ3) and the lower part of the hinge between Cgamma1 (Cγ1) and Cγ2. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, VA). For IgA, Fc polypeptide comprises immunoglobulin domains Calpha2 (Cα2) and Calpha3 (Cα3) and the lower part of the hinge between Calpha1 (Cal) and Cα2. An Fc region can be synthetic, recombinant, or generated from natural sources such as IVIg.

An “Fc region-containing polypeptide” is a polypeptide that includes all or a substantial portion of an Fc region. Examples of an Fc region-containing polypeptide preparation include, e.g., a preparation of Fc fragments, a preparation of antibody molecules, a preparation of Fc-fusion proteins (e.g., an Fc-receptor fusion protein), and a preparation of pooled, polyvalent immunoglobulin molecules (e.g., IVIg). Such an Fc region-containing polypeptide may be recombinant (e.g., a recombinant Fc fragment preparation or a recombinant antibody preparation) or naturally derived (such as IVIg).

As used herein, “glycan” is a sugar, which can be monomers or polymers of sugar residues, such as at least three sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a polypeptide, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties.

As used herein, “immune-related thrombocytopenia” refers to disorders in which there is a relative decrease of platelets in the blood caused by increased destruction of platelets by the immune system. Non-limiting examples of immune-related thrombocytopenia disorders include idiopathic thrombocytopenic purapura, neonatal alloimmune thrombocytopenia, post-transfusion purapura, and systemic lupus erythematosus related thrombocytopenia.

As used herein, “IVIg” is a preparation of pooled, polyvalent IgG, including all four IgG subgroups, extracted from plasma of at least 1,000 human donors. IVIg is approved as a plasma protein replacement therapy for immune deficient patients. The level of IVIg Fc glycan sialylation varies between about 10-20% among IVIg preparations. As used herein, the term “derived from IVIg” refers to polypeptides which result from manipulation of IVIg. For example, polypeptides purified from IVIg (e.g., enriched for sialylated IgGs, modified IVIg (e.g., IVIg IgGs enzymatically sialylated), or Fc regions of IVIg (e.g., papain digested and sialylated) are derived from IVIg.

As used herein, an “N-glycosylation site of an Fc polypeptide” refers to an amino acid residue within an Fc polypeptide to which a glycan is N-linked. In some embodiments, an Fc region contains a dimer of Fc polypeptides, and the Fc region comprises two N-glycosylation sites, one on each Fc polypeptide.

As used herein “percent (%) of branched glycans” refers to the number of moles of glycan X relative to total moles of glycans present, wherein X represents the glycan of interest.

As used herein “percent (%) sequence identity” with respect to a sequence is defined as the percentage of amino acid residues or nucleotides in a candidate sequence that are identical with the amino acid residues or nucleotides in the reference sequence, after aligning the sequences and introducing gaps, if necessary, to achieve the maximum percent sequence identity. Gaps can be introduced in one or both of a first and a second amino acid or nucleic acid sequence for optimal alignment and non-homologous sequences can be disregarded for comparison purposes. Alignment for purposes of determining percent sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. In one embodiment, the length of a reference sequence aligned for comparison purposes is at least 30%, e.g., at least 40%, e.g., at least 50%, 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. In some instances a product will include amino acid variants, e.g., species that differ at terminal residues, e.g., at one, two, three, or four N-terminal residues and/or one C-terminal residue. In instances of such cases the sequence identity which is compared is the identity between the primary amino acid sequences of the most abundant active species in each of the products being compared. In some instances sequence identity refers to the amino acid sequence encoded by a nucleic acid that can be used to make the product.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

“Pharmaceutical preparations” and “pharmaceutical products” can be included in kits containing the preparation or product and instructions for use.

“Pharmaceutical preparations” and “pharmaceutical products” generally refer to compositions in which the final predetermined level of sialylation has been achieved, and which are free of process impurities. To that end, “pharmaceutical preparations” and “pharmaceutical products” are substantially free of ST6Gal sialyltransferase and/or sialic acid donor (e.g., cytidine 5′-monophospho-N-acetyl neuraminic acid) or the byproducts thereof (e.g., cytidine 5′-monophosphate).

“Pharmaceutical preparations” and “pharmaceutical products” are generally substantially free of other components of a cell in which the glycoproteins were produced (e.g., the endoplasmic reticulum or cytoplasmic proteins and RNA), if recombinant.

As used herein, “polynucleotide” (or “nucleotide sequence” or “nucleic acid molecule”) refers to an oligonucleotide, nucleotide, or polynucleotide, and fragments or portions thereof, and to DNA or RNA of genomic or synthetic origin, which may be single- or double-stranded, and represent the sense or anti-sense strand.

As used herein, “polypeptide” (or “amino acid sequence” or “protein”) refers to a glycoprotein, oligopeptide, peptide, polypeptide, or protein sequence, and fragments or portions thereof, and to naturally occurring or synthetic molecules. “Amino acid sequence” and like terms, such as “polypeptide” or “protein,” are not meant to limit the indicated amino acid sequence to the complete, native amino acid sequence associated with the recited protein molecule.

“Predetermined level” as used herein, refers to a pre-specified particular level of one or more particular glycans, e.g., branched glycans having a sialic acid on an α1,3 arm, and/or branched glycans having a sialic acid on an α1,6 arm, and/or branched glycans having a sialic acid on an α1,3 arm and on an α1,6 arm. In some embodiments, a predetermined level is an absolute value or range. In some embodiments, a predetermined level is a relative value. In some embodiments, a predetermined level is the same as or different (e.g., higher or lower than) a level of one or more particular glycans (e.g., branched glycans having a sialic acid on an α1,3 arm, and/or branched glycans having a sialic acid on an α1,6 arm, and/or branched glycans having a sialic acid on an α1,3 arm and on an α1,6 arm) in a reference, e.g., a reference polypeptide product, or a level specified in a reference document such as a pharmaceutical specification, a monograph, alert limit, or master batch record for a pharmaceutical product.

In some embodiments, a predetermined level is an absolute level or range of (e.g., number of moles of) one or more glycans (e.g., branched glycans having a sialic acid on an α1,3 arm, and/or branched glycans having a sialic acid on an α1,6 arm, and/or branched glycans having a sialic acid on an α1,3 arm and on an α1,6 arm) in a polypeptide preparation. In some embodiments, a predetermined level is a level or range of one or more glycans (e.g., branched glycans having a sialic acid on an α1,3 arm, and/or branched glycans having a sialic acid on an α1,6 arm, and/or branched glycans having a sialic acid on an α1,3 arm and on an α1,6 arm) in a polypeptide preparation relative to total level of glycans in the polypeptide preparation. In some embodiments, a predetermined level is a level or range of one or more glycans (e.g., branched glycans having a sialic acid on an α1,3 arm, and/or branched glycans having a sialic acid on an α1,6 arm, and/or branched glycans having a sialic acid on an α1,3 arm and on an α1,6 arm) in a polypeptide preparation relative to total level of sialylated glycans in the polypeptide preparation. In some embodiments, a predetermined level is expressed as a percent.

By “purified” (or “isolated”) refers to a polynucleotide or a polypeptide that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acids. An isolated polynucleotide or polypeptide can be at least 60% free, or at least 75% free, or at least 90% free, or at least 95% free from other components present in natural environment of the indicated polynucleotide or polypeptide.

“Reference polypeptide” refers to a polypeptide having substantially the same amino acid sequence as (e.g., having about 95-100% identical amino acids of) a polypeptide described herein, e.g., a polypeptide to which it is compared. In some embodiments, a reference polypeptide is a therapeutic polypeptide described herein, e.g., an FDA approved therapeutic polypeptide.

As used herein, the term “sialylated” refers to a glycan having a terminal sialic acid. The term “mono-sialylated” refers to branched glycans having one terminal sialic acid, e.g., on an α1,3 arm or an α1,6 arm. The term “di-sialylated” refers to a branched glycan having a terminal sialic acid on two arms, e.g., both an α1,3 arm and an α1,6 arm.

As used herein, the term “ST6 sialyltransferase” refers to a polypeptide whose amino acid sequence includes at least one characteristic sequence of and/or shows at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71% or 70% identity with a protein involved in transfer of a sialic acid to a terminal galactose of a glycan through an α2,6 linkage (e.g., ST6 Gal-I). A wide variety of ST6 sialyltransferase sequences are known in the art, such as those described herein; in some embodiments, an ST6 sialyltransferase shares at least one characteristic sequence of and/or shows the specified degree of overall sequence identity with one of the ST6 sialyltransferases set forth herein (each of which may be considered a “reference” ST6 sialyltransferase). In some embodiments, an ST6 sialyltransferase as described herein shares at least one biological activity with a reference ST6 sialyltransferase as set forth herein. In some such embodiment, the shared biological activity relates to transfer of a sialic acid to a glycan.

The term “subject,” as used herein, means any subject for whom diagnosis, prognosis, or therapy is desired. In one embodiment, the subject is a human.

The term “thrombopoietin receptor agonist,” as used herein, refers to pharmaceutical agents that stimulate platelet production in the bone marrow through interaction with the thrombopoietin receptor.

The term “treatment” or “treating,” as used herein, refers to administering a therapy in an amount, manner, and/or mode effective to improve a condition, symptom, or parameter associated with a disorder or condition or to prevent or reduce progression of a disorder or condition to a degree detectable to one skilled in the art. An effective amount, manner, or mode can vary depending on the subject and may be tailored to the subject. The term “not being treated,” as used herein, means a subject is not currently being administered a therapy.

As used herein, the terms “coupled,” “linked,” “joined,” “fused,” and “fusion” are used interchangeably. These terms refer to the joining together of two more elements or components by whatever means, including chemical conjugation or recombinant means.

While the present disclosure provides exemplary units and methods for the evaluation, identification, and production methods disclosed herein, a person of ordinary skill in the art will appreciate that performance of the evaluation, identification, and production methods herein is not limited to use of those units and/or methods. For example, “percent of branched glycans” provided herein are generally described, as a value for a glycan or structure relative to total glycan or structure on a mol/mol basis. A person of skill in the art understands that although the use of other metrics or units (e.g., mass/mass, mole percent vs. weight percent) to measure a described parameter might give rise to different absolute values than those described herein, a test preparation meets a disclosed target value even if other units or metrics are used, as long as the test preparation meets the herein disclosed value when the herein disclosed units and metrics are used, e.g., allowing for the sensitivity (e.g., analytical variability) of the method being used to measure the value.

### I. Polypeptides

Examples of an Fc region-containing polypeptide preparation include, e.g., a preparation of Fc fragments, a preparation of antibody molecules, a preparation of Fc-fusion proteins (e.g., an Fc-receptor fusion protein), and a preparation of pooled, polyvalent immunoglobulin molecules (e.g., IVIg). Fc region-containing polypeptides may be recombinant or naturally derived.

Naturally derived polypeptides that can be used in the methods of the invention include, for example, intravenous immunoglobulin (IVIg) and polypeptides derived from IVIg (e.g., polypeptides purified from IVIg (e.g., enriched for sialylated IgGs), modified IVIg (e.g., IVIg IgGs enzymatically sialylated), or Fc regions of IVIg (e.g., papain digested and sialylated)).

Recombinant Fc region-containing polypeptides that can be used in the methods of the invention can be, for example, expressed in and purified from CHO cells and sialylated using human ST6-Gal sialtransferase enzyme (expressed in and purified from E. coli cells) or expressed in and purified from CHO cells and sialylated using human ST6-Gal sialtransferase enzyme (expressed in and purified from CHO cells).

### A. N-Linked Glycosylation

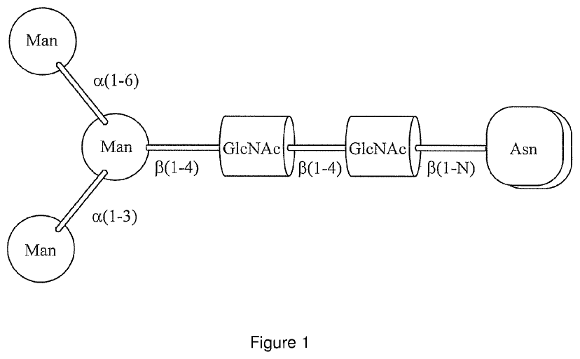

N-linked oligosaccharide chains are added to a protein in the lumen of the endoplasmic reticulum. Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains three glucose, nine mannose, and two N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues. One of the branches is referred to in the art as the “a 1,3 arm,” and the second branch is referred to as the “a 1,6 arm,” as denoted in FIG. 1.

N-glycans can be subdivided into three distinct groups called “high mannose type,” “hybrid type,” and “complex type,” with a common pentasaccharide core (Man (α 1,6)-(Man(α 1,3))-Man(β 1,4)-GlcpNAc(β 1,4)-GlcpNAc(β 1,N)-Asn) occurring in all three groups.

After initial processing in the endoplasmic reticulum, the polypeptide is transported to the Golgi where further processing may take place. If the glycan is transferred to the Golgi before it is completely trimmed to the core pentasaccharide structure, it results in a “high-mannose glycan.”

Additionally or alternatively, one or more monosaccharides units of N-acetylglucosamine may be added to the core mannose subunits to form a “complex glycan.” Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialyl residues found in polypeptides produced in mammalian expression systems are N-acetyl-neuraminic acid (NeuAc) and N-glycolylneuraminic acid (NeuGc). These usually occur as terminal structures attached to galactose (Gal) residues at the non-reducing termini of both N- and O-linked glycans. The glycosidic linkage configurations for these sialyl groups can be either α 2,3 or α 2,6.

Fc regions are glycosylated at conserved, N-linked glycosylation sites. For example, each heavy chain of an IgG antibody has a single N-linked glycosylation site at Asn297 of the C_{H}2 domain. IgA antibodies have N-linked glycosylation sites within the C_{H}2 and C_{H}3 domains, IgE antibodies have N-linked glycosylation sites within the C_{H}3 domain, and IgM antibodies have N-linked glycosylation sites within the C_{H}1, C_{H}2, C_{H}3, and C_{H}4 domains.

Each antibody isotype has a distinct variety of N-linked carbohydrate structures in the constant regions. For example, IgG has a single N-linked biantennary carbohydrate at Asn297 of the C_{H}2 domain in each Fc polypeptide of the Fc region, which also contains the binding sites for C1q and FcγR. For human IgG, the core oligosaccharide normally consists of GlcNAc₂Man₃GlcNAc, with differing numbers of outer residues. Variation among individual IgG can occur via attachment of galactose and/or galactose-sialic acid at one or both terminal GlcNAc or via attachment of a third GlcNAc arm (bisecting GlcNAc).

### B. Antibodies

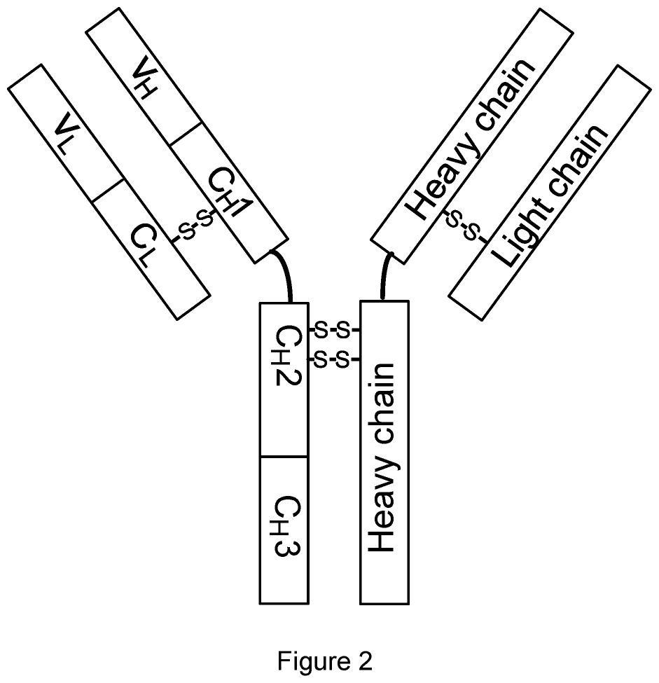

The basic structure of an IgG antibody is illustrated in FIG. 2. As shown in FIG. 2, an IgG antibody consists of two identical light polypeptide chains and two identical heavy polypeptide chains linked together by disulphide bonds. The first domain located at the amino terminus of each chain is variable in amino acid sequence, providing the antibody binding specificities found in each individual antibody. These are known as variable heavy (V_{H}) and variable light (V_{L}) regions. The other domains of each chain are relatively invariant in amino acid sequence and are known as constant heavy (C_{H}) and constant light (C_{L}) regions. As shown in FIG. 2, for an IgG antibody, the light chain includes one variable region (V_{L}) and one constant region (C_{L}). An IgG heavy chain includes a variable region (V_{H}), a first constant region (C_{H}1), a hinge region, a second constant region (C_{H}2), and a third constant region (C_{H}3). In IgE and IgM antibodies, the heavy chain includes an additional constant region (C_{H}4).

Antibodies described herein can include, for example, monoclonal antibodies, polyclonal antibodies, multispecific antibodies, human antibodies, humanized antibodies, camelized antibodies, chimeric antibodies, single-chain Fvs (scFv), disulfide-linked Fvs (sdFv), and anti-idiotypic (anti-Id) antibodies, and antigen-binding fragments of any of the above. Antibodies can be of any type (e.g., IgG, IgE, IgM, IgD, IgA, or IgY), class (e.g., IgG1, IgG2, IgG3, IgG4, IgA1, or IgA2) or subclass.

The term “Fc fragment,” as used herein, refers to one or more fragments of an Fc region that retains an Fc function and/or activity described herein, such as binding to an Fc receptor. Examples of such fragments include fragments that include an N-linked glycosylation site of an Fc region (e.g., an Asn297 of an IgG heavy chain or homologous sites of other antibody isotypes), such as a CH2 domain. The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)₂ fragment, a Fd fragment, a Fv fragment, a scFv fragment, a dAb fragment (Ward et al., (1989) Nature 341:544-546), and an isolated complementarily determining region (CDR). These antibody fragments can be obtained using conventional techniques known to those with skill in the art, and the fragments can be screened for utility in the same manner as are intact antibodies.

Reference Fc region-containing polypeptides described herein can be produced by any method known in the art for the synthesis of antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645).

Additional reference Fc region-containing polypeptides described herein are bispecific antibodies and multivalent antibodies, as described in, e.g., Segal et al., J. Immunol. Methods 248:1-6 (2001); and Tutt et al., J. Immunol. 147: 60 (1991).

### C. Polypeptide Conjugates

The disclosure includes polypeptides (or Fc regions or Fc fragments thereof containing one or more N-glycosylation sites) that are conjugated or fused to one or more heterologous moieties and that have different levels of sialylated glycans relative to a corresponding reference polypeptide. Heterologous moieties include, but are not limited to, peptides, polypeptides, proteins, fusion proteins, nucleic acid molecules, small molecules, mimetic agents, synthetic drugs, inorganic molecules, and organic molecules. In some instances, a reference polypeptide is a fusion protein that comprises a peptide, polypeptide, protein scaffold, scFv, dsFv, diabody, Tandab, or an antibody mimetic fused to an Fc region, such as a glycosylated Fc region. The fusion protein can include a linker region connecting the Fc region to the heterologous moiety (see, e.g., Hallewell et al. (1989), J. Biol. Chem. 264, 5260-5268; Alfthan et al. (1995), Protein Eng. 8, 725-731; Robinson & Sauer (1996)).

In some instances, a reference fusion protein includes an Fc region (or an Fc fragment containing one or more N-glycosylation sites thereof) conjugated to a heterologous polypeptide of at least 10, at least 20, at least 30, at least 40, at least 50, at least 60, at least 70, at least 80, at least 90, or at least 100 amino acids.

In some instances, a reference fusion protein can include an Fc region (or Fc fragment containing one or more N-glycosylation sites thereof) conjugated to marker sequences, such as a peptide to facilitate purification. A particular marker amino acid sequence is a hexa-histidine peptide, such as the tag provided in a pQE vector (QIAGEN, Inc., 9259 Eton Avenue, Chatsworth, Calif., 91311). Other peptide tags useful for purification include, but are not limited to, the hemagglutinin “HA” tag, which corresponds to an epitope derived from the influenza hemagglutinin protein (Wilson et al., 1984, Cell 37:767) and the “Flag” tag.

In other instances, a reference polypeptide (or an Fc region or Fc fragment containing one or more N-glycosylation sites thereof) is conjugated to a diagnostic or detectable agent. Such fusion proteins can be useful for monitoring or prognosing the development or progression of disease or disorder as part of a clinical testing procedure, such as determining the efficacy of a particular therapy. Such diagnosis and detection can be accomplished by coupling the polypeptide to detectable substances including, but not limited to, various enzymes, such as but not limited to horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as but not limited to iodine (¹³¹I, ¹²⁵I, ¹²³I), carbon (¹⁴C), sulfur (³⁵S), tritium (³H), indium (¹⁵In, ¹¹³In, ¹¹²In, ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵³Gd, ¹⁵⁹Gd, ¹⁴⁹Pm, ¹⁴⁰La, ¹⁶⁹Yb, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ⁵¹Cr, ⁵⁴Mn, ⁷⁵Se, ¹¹³Sn, and ¹¹⁷Sn; positron emitting metals using various positron emission tomographies, non-radioactive paramagnetic metal ions, and molecules that are radiolabelled or conjugated to specific radioisotopes.

Techniques for conjugating therapeutic moieties to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56. (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987)).

### D. Sialyltransferase Polypeptides

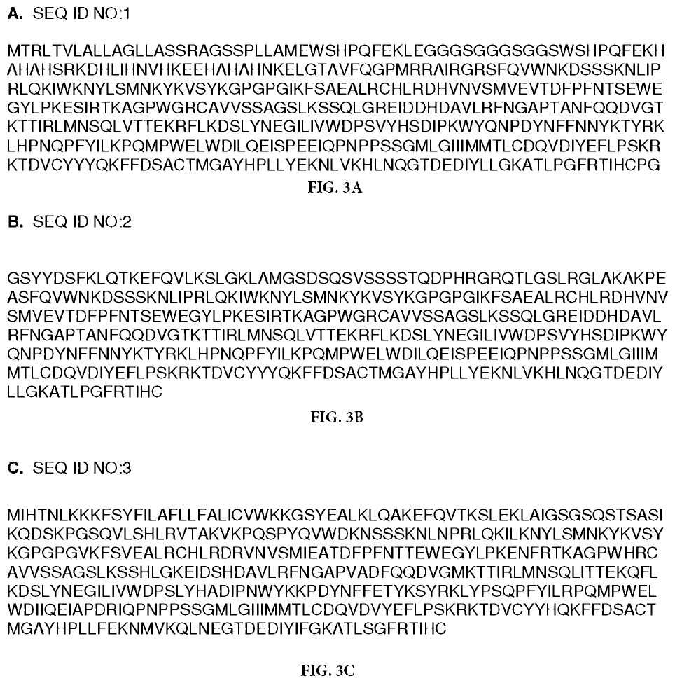

Methods and compositions described herein include the use of a sialyltransferase enzyme, e.g., an a 2,6 sialyltransferase (e.g., ST6 Gal-I). A number of ST6 sialyltransferases are known in the art and are commercially available (see, e.g., Takashima, Biosci. Biotechnol. Biochem. 72:1155-1167 (2008); Weinstein et al., J. Biol. Chem. 262:17735-17743 (1987)). ST6 Gal-I catalyzes the transfer of sialic acid from a sialic acid donor (e.g., cytidine 5′-monophospho-N-acetyl neuraminic acid) to a terminal galactose residue of glycans through an α 2,6 linkage. The sialic acid donor reaction product is cytidine 5-monophosphate. FIGS. 3A-3C depict three exemplary ST6 sialyltransferase amino acid sequences (SEQ ID NOs:1-3). In some embodiments, an ST6 sialyltransferase has or includes an amino acid sequence set forth in SEQ ID NO:1, SEQ ID NO:2, or in amino acid residues 95-416 of SEQ ID NO:3, or a characteristic sequence element thereof or therein. In some embodiments, an ST6 sialyltransferase has at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% overall sequence identity with one or more of SEQ ID NO:1, SEQ ID NO:2, or amino acid residues 95-416 of SEQ ID NO:3. Alternatively or additionally, in some embodiments, an ST6 sialyltransferase includes at least about 5, 10, 15, 20, 25, 30, 35, 40, 45, 50, 75, 100, or 150 or more contiguous amino acid residues found in SEQ ID NO:1, SEQ ID NO:2, or amino acid residues 95-416 of SEQ ID NO:3.

In some embodiments, an ST6 sialyltransferase differs from an amino acid sequence as set forth in SEQ ID NO:1, SEQ ID NO:2, or in amino acid residues 95-416 of SEQ ID NO:3, or characteristic sequence elements thereof or therein, by one or more amino acid residues. For example, in some embodiments, the difference is a conservative or nonconservative substitution of one or more amino acid residues. Conservative substitutions are those that substitute a given amino acid in a polypeptide by another amino acid of similar characteristics. Typical conservative substitutions are the following replacements: replacement of an aliphatic amino acid, such as alanine, valine, leucine, and isoleucine, with another aliphatic amino acid; replacement of a serine with a threonine or vice versa; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

In some embodiments, an ST6 sialyltransferase polypeptide includes a substituent group on one or more amino acid residues. Still other useful polypeptides are associated with (e.g., fused, linked, or coupled to) another moiety (e.g., a peptide or molecule). For example, an ST6 sialyltransferase polypeptides can be fused, linked, or coupled to an amino acid sequence (e.g., a leader sequence, a secretory sequence, a proprotein sequence, a second polypeptide, or a sequence that facilitates purification, enrichment, or stabilization of the polypeptide).

### II. Methods for Producing Sialylated Polypeptides

The present disclosure relates to Fc region-containing polypeptide preparations (e.g., IVIg, Fc, or IgG antibodies) having higher levels of branched glycans that are sialylated on an α 1,3 and 1,6 arm of the branched glycans in the Fc region (e.g., with a NeuAc-α 2,6-Gal or NeuAc-α 2,3-Gal terminal linkage), relative to a corresponding reference polypeptide preparation. The higher levels can be measured on an individual Fc region (e.g., an increase in the number of branched glycans that are sialylated on an α 1,3 arm of the branched glycans in the Fc region), or the overall composition of a preparation of polypeptides can be different (e.g., a preparation of polypeptides can have a higher number or a higher percentage of branched glycans that are sialylated on an α 1,3 arm and an α 1,6 arm of the branched glycans in the Fc region) relative to a corresponding preparation of reference polypeptides).

In exemplary methods, Fc molecules were obtained or produced from various sources, glycan compositions were characterized, and activities were determined. The Fc molecules were tested for their ability to increase reticulated platelets in immune-related thrombocytopenia models.

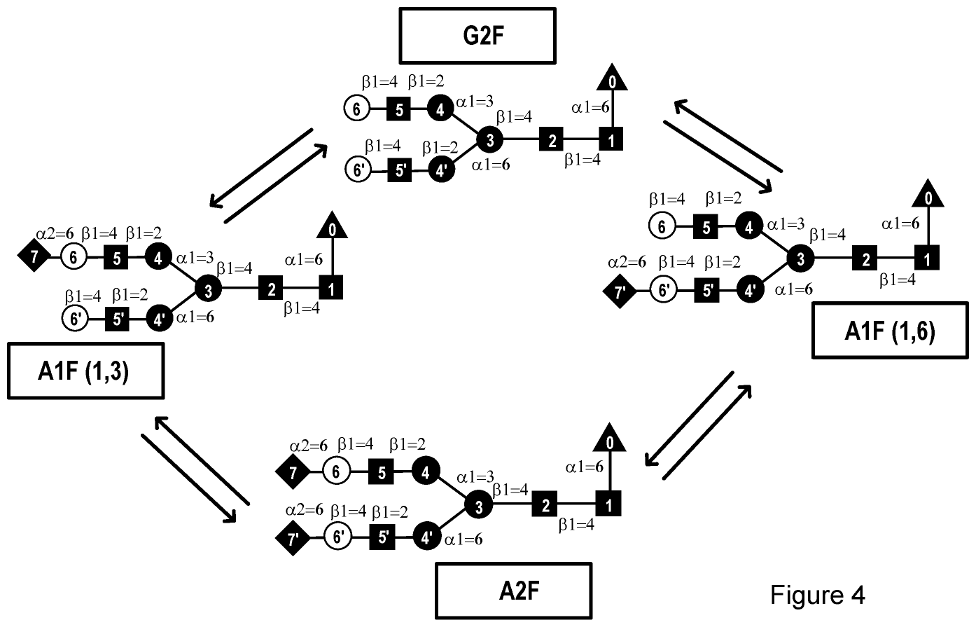

ST6 Gal-I sialyltransferase catalyzes the transfer of sialic acid from a sialic acid donor (e.g., cytidine 5′-monophospho-N-acetyl neuraminic acid) to a terminal galactose residue of glycans through an a 2,6 linkage. The present disclosure exploits the discovery that ST6 sialyltransferase catalyzes the transfer of sialic acid to branched glycans (e.g., Fc branched glycans) comprising an a 1,3 arm and an a 1,6 arm in an ordered fashion. As shown in FIG. 4, ST6 sialyltransferase transfers a sialic acid to an a 1,3 arm of a branched glycan, which can be followed by transfer of a second sialic acid to an a 1,6 arm (yielding a disialylated branched glycan), and can further be followed by removal of sialic acid from an a 1,3 arm (yielding a branched glycan having a sialic acid on an a 1,6 arm). Accordingly, by controlling and/or modulating activity (e.g., kinetics) of ST6 sialyltransferase, polypeptides having particular sialylation patterns can be produced.

Any parameter generally known to affect enzyme kinetics can be controlled and/or modulated to produce a polypeptide preparation having a predetermined level of sialic acid on an a 1,3 arm of a branched glycan, on an a 1,6 arm of a branched glycan, and/or on an a 1,3 arm and an a 1,6 arm of a branched glycan. For example, reaction time, ST6 sialyltransferase concentration and/or specific activity, branched glycan concentration, sialic acid donor concentration, sialic acid donor reaction product concentration, pH, buffer composition, and/or temperature can be controlled and/or modulated to produce a polypeptide preparation having a desired level of sialylation (e.g., a 1,3 arm and/or a 1,6 arm sialylation).

In some embodiments, to preferentially sialylate an α1,3 arm of branched glycans (e.g., having an a 1,3 arm and an a 1,6 arm), branched glycans are contacted in vitro with an ST6 sialyltransferase under limited reaction conditions. Such limited reaction conditions are selected such that addition of a sialic acid to an a 1,3 arm is enhanced relative to addition of a sialic acid to an a 1,6 arm (e.g., rate of transfer of a sialic acid to an a 1,3 arm (“Rₐ^{1,3}”) exceeds rate of transfer of a sialic acid to an a 1,6 arm (“Rₐ^{1,6}”). In some embodiments, limited reaction conditions are further selected such that removal of a sialic acid from an α1,6 arm is enhanced relative to addition of a sialic acid to an a 1,6 arm (e.g., rate of removal of a sialic acid from an a 1,6 arm (“Rᵣ^{1,6}”) exceeds rate of transfer of a sialic acid to an a 1,6 arm (“Rₐ^{1,6}”). Limited reaction conditions can include, for example, reduced reaction time, reduced enzyme concentration and/or activity, reduced amount of branched glycans, reduced level of sialic acid donor, and/or reduced temperature.

In some embodiments, to preferentially sialylate an α1,6 arm of branched glycans (e.g., having an α 1,3 arm and an a 1,6 arm), branched glycans can be contacted in vitro with an ST6 sialyltransferase under extended reaction conditions. Such extended reaction conditions are selected such that addition of a sialic acid to an α 1,6 arm is enhanced relative to removal of a sialic acid from an α 1,6 arm (e.g., rate of transfer of a sialic acid to an a 1,6 arm (“Rₐ^{1,6}”) exceeds rate of removal of a sialic acid from an a 1,6 arm (“Rᵣ^{1,6}”)). In some embodiments, extended reaction conditions are further selected such that, after initial conditions that enhance addition of sialic acid to an α 1,3 arm, conditions are extended such that removal of a sialic acid from an α 1,3 arm is eventually enhanced relative to addition of a sialic acid to an a 1,3 arm (e.g., rate of removal of a sialic acid from an α 1,3 arm (“Rᵣ^{1,3}”) exceeds rate of transfer of a sialic acid to an α 1,3 arm (“Rₐ^{1,3}”)). Extended reaction conditions can include, for example, increased reaction time, increased enzyme concentration and/or activity, increased amount of branched glycans, increased level of sialic acid donor, and/or increased temperature.

In some embodiments, to preferentially sialylate both an α 1,3 arm and an α 1,6 arm of branched glycans (e.g., having an α 1,3 arm and an α 1,6 arm), branched glycans are contacted in vitro with an ST6 sialyltransferase under intermediate reaction conditions. Such intermediate reaction conditions are selected such that addition of a sialic acid to an α 1,3 arm is enhanced relative to removal of a sialic acid from an α 1,3 arm (e.g., rate of transfer of a sialic acid to an α 1,3 arm (“Rₐ^{1,3}”) exceeds rate of removal of a sialic acid from an α 1,3 arm (“Rᵣ^{1,3}”). In some embodiments, intermediate reaction conditions are further selected such that addition of a sialic acid to an α 1,6 arm is enhanced relative to removal of a sialic acid from an α 1,6 arm (e.g., rate of addition of a sialic acid to an α 1,6 arm (“Rₐ^{1,6}”) exceeds rate of removal of a sialic acid from an a 1,6 arm (“Rᵣ^{1,6}”). Intermediate reaction conditions can include, for example, intermediate reaction time, intermediate enzyme concentration and/or activity, intermediate amount of branched glycans, intermediate level of sialic acid donor, and/or intermediate temperature. In some embodiments, intermediate reaction conditions further include supplementing the sialic acid donor at least once during the reaction. In some embodiments, intermediate reaction conditions further include removing a sialic acid donor reaction product at least once during the reaction. In some embodiments, intermediate reaction conditions further include supplementing the sialic acid donor reaction product at least once during the reaction.

In some embodiments, a polypeptide, e.g., a glycosylated antibody, is sialylated after the polypeptide is produced. For example, a polypeptide can be recombinantly expressed in a host cell (as described herein) and purified using standard methods. The purified polypeptide is then contacted with an ST6 sialyltransferase (e.g., a recombinantly expressed and purified ST6 sialyltransferase) in the presence of reaction conditions as described herein. In certain embodiments, the conditions include contacting the purified polypeptide with an ST6 sialyltransferase in the presence of a sialic acid donor, e.g., cytidine 5′-monophospho-N-acetyl neuraminic acid, manganese, and/or other divalent metal ions. In some embodiments, IVIg is used in a sialylation method described herein.

In some embodiments, chemoenzymatic sialylation is used to sialylate polypeptides. Briefly, this method involves sialylation of a purified branched glycan, followed by incorporation of the sialylated branched glycan en bloc onto a polypeptide to produce a sialylated polypeptide.

A branched glycan can be synthesized de novo using standard techniques or can be obtained from a polypeptide preparation (e.g., a recombinant polypeptide, Fc, or IVIg) using an appropriate enzyme, such as an endoglycosidase (e.g., EndoH or EndoF). After sialylation of the branched glycan, the sialylated branched glycan can be conjugated to a polypeptide using an appropriate enzyme, such as a transglycosidase, to produce a sialylated polypeptide.

In one exemplary method, a purified branched N-glycan is obtained from a polypeptide (e.g., a polypeptide preparation, e.g., IVIg) using an endoglycosidase. The purified branched N-glycan is then chemically activated on the reducing end to form a chemically active intermediate. The branched N-glycan is then further processed, trimmed, and/or glycosylated using appropriate known glycosidases. The branched glycan is then sialylated using an ST6 sialylation as described herein. After engineering, the desired branched N-glycan is transferred onto a polypeptide using a transglycosidase (such as a transglycosidase in which glycosidic activity has been attenuated using genetically engineering).

In some embodiments, a branched glycan used in methods described herein is a galactosylated branched glycan (e.g., includes a terminal galactose residue). In some embodiments, a branched glycan is galactosylated before being sialylated using a method described herein. In some embodiments, a branched glycan is first contacted with a galactosyltransferase (e.g., a beta-1,3-galactosyltransferase) and subsequently contacted with an ST6 sialyltransferase as described herein. In some embodiments, a galactosylated glycan is purified before being contacted with an ST6 sialyltransferase. In some embodiments, a galactosylated glycan is not purified before being contacted with an ST6 sialyltransferase. In some embodiments, a branched glycan is contacted with a galactosyltransferase and an ST6 sialyltransferase in a single step.

In some embodiments, a host cell is genetically engineered to express a polypeptide described herein and one or more sialyltransferase enzymes, e.g., an ST6 sialyltransferase. In some embodiments, the host cell is genetically engineered to further express a galactosyltransferase. The genetically engineered host cell can be cultured under conditions sufficient to produce a particular sialylated polypeptide. For example, to produce polypeptides preferentially sialylated on α1,3 arms of branched glycans, a host cell can be genetically engineered to express a relatively low level of ST6 sialyltransferase, whereas to produce polypeptides preferentially sialylated on α1,6 arms of branched glycans, a host cell can be genetically engineered to express a relatively high level of ST6 sialyltransferase. In some embodiments, to produce polypeptides preferentially sialylated on α1,3 arms of branched glycans, a genetically engineered host cell can be cultured in a relatively low level of sialic acid donor, whereas to produce polypeptides preferentially sialylated on α1,6 arms of branched glycans, a genetically engineered host cell can be cultured in a relatively high level of sialic acid donor.

Recombinant expression of a gene, such as a nucleic acid encoding a reference polypeptide and/or a sialtransferase described herein, can include construction of an expression vector containing a polynucleotide that encodes a reference polypeptide and/or a sialtransferase. Once a polynucleotide has been obtained, a vector for the production of the reference polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then cultured by conventional techniques to produce reference polypeptides.

A variety of host expression vector systems can be used (see, e.g., U.S. Pat. No. 5,807,715). Such host-expression systems can be used to produce polypeptides and, where desired, subsequently purified. Such host expression systems include microorganisms such as bacteria (e.g., E. coli and B. subtilis) transformed with recombinant bacteriophage DNA, plasmid DNA or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g. Ti plasmid) containing polypeptide coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For bacterial systems, a number of expression vectors can be used, including, but not limited to, the E. coli expression vector pUR278 (Ruther et al., 1983, EMBO 12:1791); pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST).

For expression in mammalian host cells, viral-based expression systems can be utilized (see, e.g., Logan & Shenk, 1984, Proc. Natl. Acad. Sci. USA 8 1:355-359). The efficiency of expression can be enhanced by the inclusion of appropriate transcription enhancer elements, transcription terminators, etc. (see, e.g., Bittner et al., 1987, Methods in Enzymol. 153:516-544).

In addition, a host cell strain can be chosen that modulates the expression of the inserted sequences, or modifies and processes the gene product in the specific fashion desired. Different host cells have characteristic and specific mechanisms for the post-translational processing and modification of proteins and gene products. Appropriate cell lines or host systems can be chosen to ensure the correct modification and processing of the polypeptide expressed. Such cells include, for example, established mammalian cell lines and insect cell lines, animal cells, fungal cells, and yeast cells. Mammalian host cells include, but are not limited to, CHO, VERY, BHK, HeLa, COS, MDCK, 293, 3T3, W138, BT483, Hs578T, HTB2, BT20 and T47D, NS0 (a murine myeloma cell line that does not endogenously produce any immunoglobulin chains), CRL7030 and HsS78Bst cells.

For long-term, high-yield production of recombinant proteins, host cells are engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

In some embodiments, a reference Fc region-containing polypeptide is recombinantly produced in cells as described herein, purified, and contacted with a sialtransferase enzyme in vitro to produce Fc region-containing polypeptides containing higher levels of glycans having higher levels of sialic acid on the α 1,3 arms and α 1,6 arms of the branched glycans with a NeuAc-α 2,6-Gal terminal linkage, relative to the reference polypeptide. In some embodiments, a purified reference polypeptide is contacted with the sialtransferase in the presence of CMP-sialic acid, manganese, and/or other divalent metal ions.

A reference Fc region-containing polypeptide can be purified by any method known in the art for purification, for example, by chromatography (e.g., ion exchange, affinity, and sizing column chromatography), centrifugation, differential solubility, or by any other standard technique for the purification of proteins. For example, a reference antibody can be isolated and purified by appropriately selecting and combining affinity columns such as Protein A column with chromatography columns, filtration, ultra filtration, salting-out and dialysis procedures (see Antibodies: A Laboratory Manual, Ed Harlow, David Lane, Cold Spring Harbor Laboratory, 1988). Further, as described herein, a reference polypeptide can be fused to heterologous polypeptide sequences to facilitate purification.

In some embodiments, a polypeptide can be purified using a lectin column by methods known in the art (see, e.g., WO 02/30954). For example, a preparation of polypeptides can be enriched for polypeptides containing glycans having sialic acids in α 2,6 linkage as described in, e.g., WO2008/057634. Following enrichment of polypeptides containing glycans having sialic acids in α 2,6 linkage, the glycan composition of such polypeptides can be further characterized to identify polypeptides having sialic acids attached to the a 1,3 arm and α 1,6 arm of a branched glycan. Preparations of polypeptides containing a predetermined level of glycans having sialic acids in α 2,6 linkage on the α 1,3 arm and α 1,6 arm can be selected for use, e.g., for therapeutic use. Such compositions can have increased levels of anti-inflammatory activity.

In accordance with the present disclosure, there may be employed conventional molecular biology, microbiology, and recombinant DNA techniques within the skill of the art. Such techniques are described in the literature (see, e.g., Green & Sambrook, Molecular Cloning: A Laboratory Manual, Fourth Edition (2012) Cold Spring Harbor Laboratory Press, Cold Spring Harbor, N.Y.; DNA Cloning: A Practical Approach, Volumes I and II (Glover and Hames, eds. 1995); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); R. I. Freshney, Culture of Animal Cells: A Manual of Basic Technique and Specialized Application (2010); Immobilized Cells and Enzymes (IRL Press, (1986)); J. M. Guisan, Immobilization of Enzymes and Cells (2013); B. Perbal, A Practical Guide To Molecular Cloning (1984); T. A. Brown, Essential Molecular Biology: A Practical Approach Volume I (2000); T. A. Brown, Essential Molecular Biology: A Practical Approach Volume II (2002); F. M. Ausubel et al. (eds.), Current Protocols in Molecular Biology, John Wiley & Sons, Inc. (1994).

Glycan compositions can be characterized using methods described in, e.g., Barb, Biochemistry 48:9705-9707 (2009); Anumula, J. Immunol. Methods 382:167-176 (2012); Gilar et al., Analytical Biochem. 417:80-88 (2011).

### Glycan Evaluation

Glycans of polypeptides can be evaluated using any methods known in the art. For example, sialylation of glycan compositions (e.g., level of branched glycans that are sialylated on an α1,3 arm and/or an α1,6 arm) can be characterized using methods described in, e.g., Barb, Biochemistry 48:9705-9707 (2009); Anumula, J. Immunol. Methods 382:167-176 (2012); Gilar et al., Analytical Biochem. 417:80-88 (2011); Wuhrer et al., J. Chromatogr. B. 849:115-128 (2007). In some embodiments, in addition to evaluation of sialylation of glycans, one or more parameters described in Table 1 are evaluated.

In some instances, glycan structure and composition as described herein are analyzed, for example, by one or more, enzymatic, chromatographic, mass spectrometry (MS), chromatographic followed by MS, electrophoretic methods, electrophoretic methods followed by MS, nuclear magnetic resonance (NMR) methods, and combinations thereof. Exemplary enzymatic methods include contacting a polypeptide preparation with one or more enzymes under conditions and for a time sufficient to release one or more glycan(s) (e.g., one or more exposed glycan(s)). In some instances, the one or more enzymes include(s) PNGase F. Exemplary chromatographic methods include, but are not limited to, Strong Anion Exchange chromatography using Pulsed Amperometric Detection (SAX-PAD), liquid chromatography (LC), high performance liquid chromatography (HPLC), ultra performance liquid chromatography (UPLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof. Exemplary nuclear magnetic resonance (NMR) include, but are not limited to, one-dimensional NMR (1 D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

In some instances, techniques described herein may be combined with one or more other technologies for the detection, analysis, and or isolation of glycans or polypeptides. For example, in certain instances, glycans are analyzed in accordance with the present disclosure using one or more available methods (to give but a few examples, see Anumula, Anal. Biochem., 350(1):1, 2006; Klein et al., Anal. Biochem., 179:162, 1989; and/or Townsend, R. R. Carbohydrate Analysis” High Performance Liquid Chromatography and Capillary Electrophoresis, Ed. Z. El Rassi, pp 181-209, 1995; WO2008/128216; WO2008/128220; WO2008/128218; WO2008/130926; WO2008/128225; WO2008/130924; WO2008/128221; WO2008/128228; WO2008/128227; WO2008/128230; WO2008/128219; WO2008/128222; WO2010/071817; WO2010/071824; WO2010/085251; WO2011/069056; and WO2011/127322, each of which is incorporated herein by reference in its entirety). For example, in some instances, glycans are characterized using one or more of chromatographic methods, electrophoretic methods, nuclear magnetic resonance methods, and combinations thereof. In some instances, methods for evaluating one or more target protein specific parameters, e.g., in a polypeptide preparation, e.g., one or more of the parameters disclosed herein, can be performed by one or more of following methods.

TABLE 1
Exemplary methods of evaluating parameters:

| Method(s) | Relevant literature | Parameter |
|---|---|---|
| C18 UPLC Mass Spec.* | Chen and Flynn, Anal. Biochem., | Glycan(s) |
| | 370: 147-161 (2007) | (e.g., N-linked glycan, exposed N-linked |
| | Chen and Flynn, J. Am. Soc. Mass | glycan, glycan detection, glycan |
| | Spectrom., 20: 1821-1833 (2009) | identification, and characterization; site |
| | | specific glycation; glycoform detection |
| | | (e.g., parameters 1-7); percent |
| | | glycosylation; and/or aglycosyl) |
| Peptide LC-MS | Dick et al., Biotechnol. Bioeng., | C-terminal lysine |
| (reducing/non-reducing) | 100: 1132-1143 (2008) | |
| | Yan et al., J. Chrom. A., 1164: 153-161 | |
| | (2007) | |
| | Chelius et al., Anal. Chem., 78: 2370- | |
| | 2376 (2006) | |
| | Miller et al., J. Pharm. Sci., 100: 2543- | |
| | 2550 (2011) | |
| LC-MS (reducing/non- | Dick et al., Biotechnol. Bioeng., | |
| reducing/alkylated) | 100: 1132-1143 (2008) | |
| | Goetze et al., Glycobiol., 21: 949-959 | |
| | (2011) | |
| Weak cation exchange | Dick et al., Biotechnol. Bioeng., | |
| (WCX) chromatography | 100: 1132-1143 (2008) | |
| LC-MS (reducing/non- | Dick et al., Biotechnol. Bioeng., | N-terminal pyroglu |
| reducing/alkylated) | 100: 1132-1143 (2008) | |
| | Goetze et al., Glycobiol., 21: 949-959 | |
| | (2011) | |
| PeptideLC- | Yan et al., J. Chrom. A., 1164: 153-161 | |
| MS (reducing/non- | (2007) | |
| reducing) | Chelius et al., Anal. Chem., 78: 2370- | |
| | 2376 (2006) | |
| | Miller et al., J. Pharm. Sci., 100: 2543- | |
| | 2550 (2011) | |
| Peptide LC-MS | Yan et al., J. Chrom. A., 1164: 153-161 | Methionine oxidation |
| (reducing/non-reducing) | (2007); | |
| | Xie et al., mAbs, 2: 379-394 (2010) | |
| Peptide LC-MS | Miller et al., J. Pharm. Sci., 100: 2543- | Site specific glycation |
| (reducing/non-reducing) | 2550 (2011) | |
| Peptide LC-MS | Wang et al., Anal. Chem., 83: 3133-3140 | Free cysteine |
| (reducing/non-reducing) | (2011); | |
| | Chumsae et al., Anal. Chem., 81: 6449- | |
| | 6457 (2009) | |
| Bioanalyzer | Forrer et al., Anal. Biochem., 334: 81-88 | Glycan (e.g., N-linked glycan, exposed N- |
| (reducing/non-reducing)* | (2004) | linked glycan) |
| | | (including, for example, glycan detection, |
| | | identification, and characterization; site |
| | | specific glycation; glycoform detection; |
| | | percent glycosylation; and/or aglycosyl) |
| LC-MS (reducing/non- | Dick et al., Biotechnol. Bioeng., | Glycan (e.g., N-linked glycan, exposed N- |
| reducing/alkylated)* | 100: 1132-1143 (2008) | linked glycan) |
| * Methods include | Goetze et al., Glycobiol., 21: 949-959 | (including, for example, glycan detection, |
| removal (e.g., enzymatic, | (2011) | identification, and characterization; site |
| chemical, and | Xie et al., mAbs, 2: 379-394 (2010) | specific glycation; glycoform detection; |
| physical) of glycans | | percent glycosylation; and/or aglycosyl) |
| Bioanalyzer | Forrer et al., Anal. Biochem., 334: 81-88 | Light chain: Heavy chain |
| (reducing/non-reducing) | (2004) | |
| Peptide LC-MS | Yan et al., J. Chrom. A., 1164: 153-161 | Non-glycosylation-related peptide |
| (reducing/non-reducing) | (2007) | modifications (including, for example, |
| | Chelius et al., Anal. Chem., 78: 2370- | sequence analysis and identification of |
| | 2376 (2006) | sequence variants; |
| | Miller et al., J. Pharm. Sci., 100: 2543- | oxidation; succinimide; aspartic acid; |
| | 2550 (2011) | and/or site-specific aspartic acid) |
| Weak cation exchange | Dick et al., Biotechnol. Bioeng., | Isoforms (including, for example, charge |
| (WCX) chromatography | 100: 1132-1143 (2008) | variants (acidic variants and basic |
| | | variants); and/or deamidated variants) |
| Anion-exchange | Ahn et al., J. Chrom. B, 878: 403-408 | Sialylated glycan |
| chromatography | (2010) | |
| Anion-exchange | Ahn et al., J. Chrom. B, 878: 403-408 | Sulfated glycan |
| chromatography | (2010) | |
| 1,2-diamino-4,5- | Hokke et al., FEBS Lett., 275: 9-14 | Sialic acid |
| methylenedioxybenzene | (1990) | |
| (DMB) labeling method | | |
| LC-MS | Johnson et al., Anal. Biochem., 360: 75- | C-terminal amidation |
| | 83 (2007) | |
| LC-MS | Johnson et al., Anal. Biochem., 360: 75- | N-terminal fragmentation |
| | 83 (2007) | |
| Circular dichroism | Harn et al., Current Trends in | Secondary structure (including, for |
| spectroscopy | Monoclonal Antibody Development and | example, alpha helix content and/or beta |
| | Manufacturing, S. J. Shire et al., eds, | sheet content) |
| | 229-246 (2010) | |
| Intrinsic and/or ANS dye | Harn et al., Current Trends in | Tertiary structure (including, for example, |
| fluorescence | Monoclonal Antibody Development and | extent of protein folding) |
| | Manufacturing, S. J. Shire et al., eds, | |
| | 229-246 (2010) | |
| Hydrogen-deuterium | Houde et al., Anal. Chem., 81: 2644- | Tertiary structure and dynamics (including, |
| exchange-MS | 2651 (2009) | for example, accessibility of amide protons |
| | | to solvent water) |
| Size-exclusion | Carpenter et al., J. Pharm. Sci., | Extent of aggregation |
| chromatography | 99: 2200-2208 (2010) | |
| Analytical | Pekar and Sukumar, Anal. Biochem., | |
| ultracentrifugation | 367: 225-237 (2007) | |

References listed in Table 1 are hereby incorporated by reference in their entirety or, in the alternative, to the extent that they pertain to one or more of the methods disclosed in Table 1. Other methods for evaluating one or more parameters are disclosed in the examples.

III. Treatment of immune-related thrombocytopenia

The inventors have discovered that biological activity of Fc-containing molecules is enhanced by sialylation of two branches of branched glycans. Accordingly, Fc region-containing polypeptides described herein (e.g., Fc region-containing polypeptides containing glycans containing sialic acid on an α 1,3 arm and an α 1,6 arm of branched glycans with a NeuAc-α 2,6-Gal terminal linkage) have increased activity relative to a reference polypeptide. Current treatments for immune-related thrombocytopenia include IVIg infusions, platelets transfusions, and treatment with thrombopoietin or thrombopoietin receptor agonist, e.g., romiplostim (NPLATE@, Amgen) and eltrombopag (PROMACTA@, GlaxoSmithKline).

### IV. Pharmaceutical Compositions and Administration

A polypeptide of the present disclosure, e.g., an Fc region-containing polypeptide comprising branched glycans that are sialylated on both an α 1,3 arm and an α 1,6 arm of the branched glycan in the Fc region, e.g., with a NeuAc-α 2,6-Gal terminal linkage, can be incorporated into a pharmaceutical composition and can be useful in the treatment of immune-related thrombocytopenia. Such a pharmaceutical composition is useful as an improved composition for the prevention and/or treatment of diseases relative to the corresponding reference polypeptide. Pharmaceutical compositions comprising a polypeptide can be formulated by methods known to those skilled in the art. The pharmaceutical composition can be administered parenterally in the form of an injectable formulation comprising a sterile solution or suspension in water or another pharmaceutically acceptable liquid. For example, the pharmaceutical composition can be formulated by suitably combining the sulfated polypeptide with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in the pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Non-limiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampoule.

Route of administration can be parenteral, for example, administration by injection, transnasal administration, transpulmonary administration, ortranscutaneous administration. Administration can be systemic or local by intravenous injection, intramuscular injection, intraperitoneal injection, subcutaneous injection.

The term “subcutaneous administration” refers to introduction of a drug under the skin of an animal or human patient (e.g., by subcutaneous infusion or subcutaneous bolus), preferably within a pocket between the skin and underlying tissue, by relatively slow, sustained delivery from a drug receptacle. The pocket may be created by pinching or drawing the skin up and away from underlying tissue. In particular embodiments, an extracellular matrix degrading enzyme (e.g., a hyaluronidase or any extracellular matrix degrading enzyme described herein) is administered at each of the sites (e.g., prior to administration of the composition and/or during the non-delivery period). In particular embodiments, the extracellular matrix degrading enzyme is co-infused with the composition.

Convenient sites for subcutaneous administration include the shoulder, upper arm, thigh, and abdomen. In particular embodiments of the above methods, the composition is administered into subcutis or fat at a depth between 2 mm and 10 mm below the dermis of the subject.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is preferably less than approximately 15 minutes, more preferably less than 5 minutes, and most preferably less than 60 seconds. Administration is preferably within a pocket between the skin and underlying tissue, where the pocket is created, for example, by pinching or drawing the skin up and away from underlying tissue.

The term “extracellular matrix degrading enzyme” means an enzyme that can break down extracellular matrix at the site of infusion, resulting in improved tissue permeability for an composition infused at the site. Extracellular matrix degrading enzymes include enzymes catalyzing the hydrolysis of hyaluronic acid (hyaluronan), a glycosaminoglycan, chondroitin, or collagen, such as a hyaluronidase, glycosaminoglycanase, collagenase (e.g. cathepsin), serine proteases, thiol proteases, and matrix metalloproteases, of which the human enzymes are preferred and the recombinant human enzymes are most preferred. Examples of such enzymes which can be used in the methods and compositions of the invention are described in U.S. Pat. Nos. 4,258,134; 4,820,516; 7,871,607; 7,767,429; 7,829,081; 7,846,431; 7,871,607; 8,187,855; and 8,105,586, and U.S. Patent Publication Nos. 20090304665; 20110053247; 20120101325; and 20110008309, each of which is incorporated by reference. Human hyaluronidases which can be used in the methods and compositions of the invention are also described, for example, in U.S. Pat. Nos. 3,945,889; 6,057,110; 5,958,750; 5,854,046; 5,827,721; and 5,747,027, each of which is incorporated herein by reference. Commercially available hyaluronidases which can be used in the methods and compositions of the invention include HYDASE™ (PrimaPharm Inc.), VITRASSE® (ISTA Pharmaceuticals), AMPHADASE® (Amphastar Pharmaceuticals), and HYLENEX® (sold by Halozyme Therapeutics).

A suitable means of administration can be selected based on the age and condition of the patient. A single dose of the pharmaceutical composition containing a modified polypeptide can be selected from a range of 0.001 to 1000 mg/kg of body weight. On the other hand, a dose can be selected in the range of 0.001 to 100000 mg/body weight, but the present disclosure is not limited to such ranges. The dose and method of administration varies depending on the weight, age, condition, and the like of the patient, and can be suitably selected as needed by those skilled in the art.

### EXAMPLES

### Example 1—Preparation of Sialylated Glycoproteins

The sialylation of IVIg by the sialyltransferase ST6 was analyzed. IVIg was first galactosylated and then sialylated. The reactions were performed sequentially. There was no purification between galactosylation and sialylation reactions. The relative abundance of glycoforms was analyzed following the sialylation reactions.

### Galactosylation

A reaction was set up that contained the following components at the concentrations indicated in Table 2:

TABLE 2
Galactosylation conditions (Target s2IVIG)

| Constituent | Final concentration | |
|---|---|---|
| MOPS (pH 7.4) | 50 | mM |
| MnCl₂ | 8 | mM |
| IVIg | 125 | mg/ml |
| B4GaIT1 (100 u/ml) | 1.04 | mg/g-IVIG |
| UDP-Galactose | 5 | mM |

The reaction was incubated for 24-72 hours at 37° C.

### B. Sialylation

To an aliquot of the galactosylation reaction were added CMP-NANA, MOPS buffer and ST6Gal1.

The final volume was adjusted so that the final concentration of components in the reaction was as indicated in Table 3.

TABLE 3
Sialylation conditions

| Constituent | Final concentration | |
|---|---|---|
| MOPS (pH 7.4) | 50 | mM |
| IVIg | 115 | mg/ml |
| CMP-NANA (6 × 8 mM) | 48 | mM |
| ST6GaI1 (SEQ ID NO: 1) | 3.5 | mg/g-IVIg |

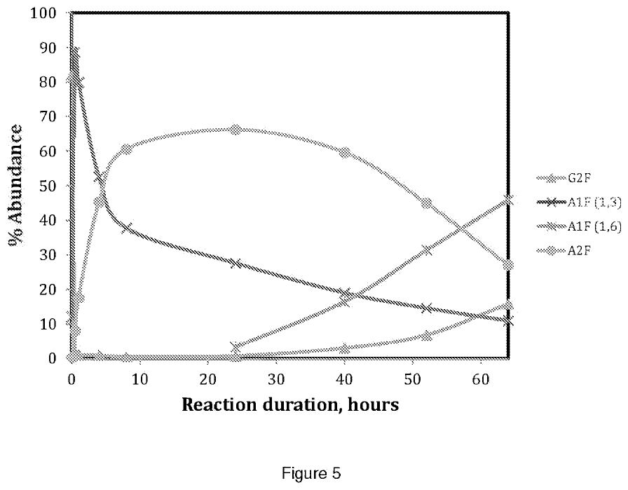

The reaction was incubated at 37° C. Aliquots were extracted at the times indicated in FIG. 5 and frozen at −20° C. for later analyses.

### C. Results

As shown in FIG. 5, the predominant glycoform changed over time from G2F to A1F (1,3) to A2F to A1F (1,6). The results are summarized in the reaction scheme depicted in FIG. 4. As shown in FIG. 4, the product glycoform can change between G2F, A1F (1,3), A2F, and A1F (1,6) during the course of a reaction due to competing addition (forward reaction) and removal (back reaction) steps.

The sialyltransferase ST6 can add sialic acid to either branch of a substrate's biantennary N-glycan. However, these results demonstrate that addition to each branch happens at different rates, resulting in different end products depending on the reaction conditions. Addition of sialic acid to the α1,3 branch is faster than addition to the α1,6 branch.

These data also demonstrate that sialyltransferase ST6 can also catalyze the removal of sialic acids from N-glycans. The removal of sialic acid from the α1,3 branch is faster than removal from the α1,6 branch. This can surprisingly lead to the production of Fc glycans substantially or primarily monosialylated on the α1,6 branch by modulating reaction conditions.

This Example demonstrates that reaction conditions can be controlled to produce a glycoprotein product having a predetermined or target sialylation levels. Such conditions can include time, ST6 sialyltransferase concentration, substrate concentration, donor sugar nucleotide concentration, product nucleotide concentration, pH, buffer composition, and/or temperature.

### Example 2—Dose Response of IVIg, S1-IVIg, S2-IVIg, and Des-IVIg in a Chronic ITP Mouse Model

The effect of IVIg, S1-IVIg, S2-IVIg, and Des-IVIg at varying doses in an anti-CD41 antibody mediated ITP mouse model was analyzed.

### A. Study Design

Sixty-six to seventy two mice were given 1.5 μg/mouse of rat anti-CD41 antibody (Ab) clone MWReg30 (BioLegend cat #133910) once daily for 4 days (on Days 1, 2, 3 and 4), intraperitoneally. Six to twelve mice were dosed in the same manner with a rat IgG1, k isotype control (BioLegend cat #400414). All mice were dosed once intravenously with saline control, IVIg, S1-IVIg, S2-IVIg, or desialylated-IVIg (Des-IVIg) at different doses 1 to 2 hours after the third anti-CD41 Ab injection (Table 4). Mice were bled on Day 4 (4 h after the forth anti-CD41 injection) and on Day 5 (24 h after the forth anti-CD41 Ab injection) to quantitate total platelet and reticulated platelet levels. To confirm that platelet depletion was successful, a subgroup of mice was bled on Day 3, prior to treatment.

TABLE 4
IVIg, S1-IVIg, S2-IVIg, and Des-IVIg dose response study details

| | | Induction (1.5 μg IP) | | | | | |
|---|---|---|---|---|---|---|---|
| Group # | n | 4 daily doses | Treatment Agent (200 uL IV) | Dose | | Timing of Dosing | Blood Sampling |
| 1 | 6 | anti-CD41 | Saline | 200 | μL | 1-2 h post 3. anti-CD41 dose | Day 3, 4 and Day 5 |
| 2 | 6 | Rat IgG1 | Saline | 200 | μL | 1-2 h post 3. anti-CD41 dose | Day 4 and Day 5 |
| 3 | 8 | anti-CD41 | IVIg Gammagard | 0.5 | g/kg | 1-2 h post 3. anti-CD41 dose | Day 4 and Day 5 |
| 4 | 8 | anti-CD41 | IVIg Gammagard | 1 | g/kg | 1-2 h post 3. anti-CD41 dose | Day 4 and Day 5 |
| 5 | 8 | anti-CD41 | S1-IVIg | 0.5 | g/kg | 1-2 h post 3. anti-CD41 dose | Day 4 and Day 5 |
| 6 | 8 | anti-CD41 | S1-IVIg | 1 | g/kg | 1-2 h post 3. anti-CD41 dose | Day 4 and Day 5 |
| 7 | 8 | anti-CD41 | S2-IVIg | 0.5 | g/kg | 1-2 h post 3. anti-CD41 dose | Day 4 and Day 5 |
| 8 | 8 | anti-CD41 | S2-IVIg | 1 | g/kg | 1-2 h post 3. anti-CD41 dose | Day 4 and Day 5 |
| 9 | 8 | anti-CD41 | Des-IVIg | 0.5 | g/kg | 1-2 h post 3. anti-CD41 dose | Day 4 and Day 5 |
| 10 | 8 | anti-CD41 | Des-IVIg | 1 | g/kg | 1-2 h post 3. anti-CD41 dose | Day 4 and Day 5 |

### B. Methods

### ITP Induction in Mice:

In vivo studies were conducted using female C57BL/6 mice (18-22 g, Charles Rivers Labs, MA). All procedures were performed in compliance with the Animal Welfare Act and with the Guide for the Care and Use of Laboratory Animals.

### Quantitation of Total Platelets:

Blood samples were collected by submandibular bleed into EDTA coated tubes, and then run on a VetScan Instrument for platelet level determination. Total platelet levels were analyzed using One-Way ANOVA with Dunnett's or Bonferroni's post-test.

### Quantitation of Reticulated Platelets:

To evaluate and quantitate for the presence of reticulated (young) platelets which contain residual RNA, whole blood was sequentially stained for total platelets (anti-CD61) followed by staining for the RNA with thiazole orange (RNA-binding dye, commercially available as ReticCount Reagent from BD Biosciences). This analysis was performed for blood samples collected on Day 5.

Ten microliters of whole blood was transferred into the bottom of α 5 mL FACS tube. Five microliters of anti-mouse CD61-PE antibody (BD Biosciences) was added directly to the whole blood and samples were mixed thoroughly by pipetting. Samples were incubated at room temperature for 5 minutes in the dark. Two milliliters of ReticCount reagent (BD Biosciences) was added to each sample and samples incubated for a minimum of 30 minutes at room temperature.

Samples were acquired on a FACS Canto flow cytometer (BD Biosciences). Total platelets were identified by forward and side scatter characteristics of the cells and distinguished from erythrocytes by gating on CD61-PE positive events. A total 10,000 platelet events were recorded for each sample. Using FlowJo software, a gate was set on the reticulated platelets (CD61 positive and thiazole orange positive) using samples from isotype control treated mice to achieve a rate of 6-10% reticulated platelets (normal rate). The same gate was then applied to all subsequent samples and treatment groups to calculate percentages of reticulated and non-reticulated platelets. Total counts of reticulated and non-reticulated platelets for each sample were calculated by multiplying the total number of platelets measured in the VetScan Instrument by the percentage of the platelet fraction. Total reticulated and non-reticulated platelet levels were analyzed using One-Way ANOVA with Dunnett's or Bonferroni's post-test.

In addition to the overall platelet quantification using the VetScan Instrument, numbers of reticulated platelets were also determined using ReticCount, anti-CD61-PE labeled Ab and flow cytometry.

### C. Results

The results of the IVIg, S1-IVIg, S2-IVIg, and Des-IVIg dose response study are shown in Table 5.

TABLE 5
Total, reticulated, and non-reticulated platelet counts (10⁹/L) on Day 5

| | Disease Induction | | | | | | | | | |
|---|---|---|---|---|---|---|---|---|---|---|
| | Isotype | | | | | | | | | |
| | Control | | | | | | | | | |
| | (1.5 μg) | Anti-CD41 Ab (1.5 μg) | | | | | | | | |
| | Treatment | | | | | | | | | |
| | | | IVIg | | S1-IVIg | | S2-IVIg | | desialylated IVIg | |
| | | | Dose | | | | | | | |
| | Saline | Saline | 0.5 g/kg | 1 g/kg | 0.5 g/kg | 1 g/kg | 0.5 g/kg | 1 g/kg | 0.5 g/kg | 1 g/kg |
| n per group | 6 | 6 | 8 | 7 | 6 | 8 | 7 | 8 | 8 | 7 |
| Total | 733 ± 73 | 240 ± 200 | 282 ± 79 | 429 ± 116 | 355 ± 137 | 356 ± 89 | 362 ± 94 | 501 ± 102 | 224 ± 51 | 182 ± 78 |
| Platelets × | | | | | | | | | | |
| 10⁹/L[mean ± | | | | | | | | | | |
| SD] | | | | | | | | | | |
| Reticulated | 70 ± 18 | 97 ± 24 | 109 ± 76 | 268 ± 84 | 196 ± 64 | 234 ± 81 | 304 ± 73 | 391 ± 86 | 175 ± 42 | 134 ± 56 |
| Platelets × | | | | | | | | | | |
| 10⁹/L [mean ± | | | | | | | | | | |
| SD] | | | | | | | | | | |
| Non- | 663 ± 64 | 143 ± 182 | 173 ± 49 | 161 ± 77 | 159 ± 85 | 122 ± 63 | 58 ± 41 | 111 ± 69 | 49 ± 32 | 48 ± 26 |
| Reticulated | | | | | | | | | | |
| Platelets × | | | | | | | | | | |
| 10⁹/L [mean ± | | | | | | | | | | |
| SD] | | | | | | | | | | |

### Example 3—Comparison of IVIg, S1-IVIg, S2-IVIg, and Des-IVIg in a Chronic ITP Mouse Model

The effect of IVIg, S1-IVIg, S2-IVIg, and Des-IVIg in an anti-CD41 antibody mediated ITP mouse model was analyzed.

### A. Study Design

Sixty-six to seventy two mice were given 1.5 μg/mouse of rat anti-CD41 antibody (Ab) clone MWReg30 (BioLegend cat #133910) once daily for 4 days (on Days 1, 2, 3 and 4), intraperitoneally. Six to twelve mice were dosed in the same manner with a rat IgG1, k isotype control (BioLegend cat #400414). All mice were dosed once intravenously with saline control, IVIg, S1-IVIg, S2-IVIg, or desialylated-IVIg (Des-IVIg at different doses 1 to 2 hours after the third anti-CD41 Ab injection (Table 6). Mice were bled on Day 4 (4 h after the forth anti-CD41 injection) and on Day 5 (24 h after the forth anti-CD41 Ab injection) to quantitate total platelet and reticulated platelet levels. To confirm that platelet depletion was successful, a subgroup of mice was bled on Day 3, prior to treatment. On Day 4 bone marrow cells were isolated to quantitate megakaryocytes.

TABLE 6
IVIg, S1-IVIg, S2-IVIg, and Des-IVIg comparison study details

| | | Induction (1.5 μg IP) | | | | | |
|---|---|---|---|---|---|---|---|
| Group # | n | 4 daily doses | Treatment Agent (200 uL IV) | Dose | | Timing of Dosing | Blood Sampling |
| 1 | 12 | anti-CD41 | Saline | 200 | μL | 1-2 h post 3. anti-CD41 dose | Day 3, Day 4, and Day 5 |
| 2 | 12 | Rat IgG1 | Saline | 200 | μL | 1-2 h post 3. anti-CD41 dose | Day 4 and Day 5 |
| 3 | 12 | anti-CD41 | IVIg Gammagard | 1 | g/kg | 1-2 h post 3. anti-CD41 dose | Day 4 and Day 5 |
| 4 | 12 | anti-CD41 | S1-IVIg | 1 | g/kg | 1-2 h post 3. anti-CD41 dose | Day 4 and Day 5 |
| 5 | 12 | anti-CD41 | S2-IVlg | 1 | g/kg | 1-2 h post 3. anti-CD41 dose | Day 4 and Day 5 |
| 6 | 12 | anti-CD41 | Des-IVIg | 1 | g/kg | 1-2 h post 3. anti-CD41 dose | Day 4 and Day 5 |

### B. Methods

### ITP Induction in Mice:

In vivo studies were conducted using female C57BL/6 mice (18-22 g, Charles Rivers Labs, MA). All procedures were performed in compliance with the Animal Welfare Act and with the Guide for the Care and Use of Laboratory Animals.

### Quantitation of Total Platelets:

Blood samples were collected by submandibular bleed into EDTA coated tubes, and then run on a VetScan Instrument for platelet level determination. Total platelet levels were analyzed using One-Way ANOVA with Dunnett's or Bonferroni's post-test.

### Quantitation of Reticulated Platelets:

To evaluate and quantitate for the presence of reticulated (young) platelets which contain residual RNA, whole blood was sequentially stained for total platelets (anti-CD61) followed by staining for the RNA with thiazole orange (RNA-binding dye, commercially available as ReticCount Reagent from BD Biosciences). This analysis was performed for blood samples collected on Day 5.

Ten microliters of whole blood was transferred into the bottom of α 5 mL FACS tube. Five microliters of anti-mouse CD61-PE antibody (BD Biosciences) was added directly to the whole blood and samples were mixed thoroughly by pipetting. Samples were incubated at room temperature for 5 minutes in the dark. Two milliliters of ReticCount reagent (BD Biosciences) was added to each sample and samples incubated for a minimum of 30 minutes at room temperature.

Samples were acquired on a FACS Canto flow cytometer (BD Biosciences). Total platelets were identified by forward and side scatter characteristics of the cells and distinguished from erythrocytes by gating on CD61-PE positive events. A total 10,000 platelet events were recorded for each sample. Using FlowJo software, a gate was set on the reticulated platelets (CD61 positive and thiazole orange positive) using samples from isotype control treated mice to achieve a rate of 6-10% reticulated platelets (normal rate). The same gate was then applied to all subsequent samples and treatment groups to calculate percentages of reticulated and non-reticulated platelets. Total counts of reticulated and non-reticulated platelets for each sample were calculated by multiplying the total number of platelets measured in the VetScan Instrument by the percentage of the platelet fraction. Total reticulated and non-reticulated platelet levels were analyzed using One-Way ANOVA with Dunnett's or Bonferroni's post-test.

In addition to the overall platelet quantification using the VetScan Instrument, numbers of reticulated platelets were also determined using ReticCount, anti-CD61-PE labeled Ab and flow cytometry.

### Quantitation of Megakaryocytes in the Bone Marrow:

On Day 4 (1 day after IVIg treatment and 4 h after the 4^{th} anti-CD41 antibody injection) of the study, bone marrow was extracted from one femur per mouse by using a syringe with α 25 gauge needle, flushing the bone shaft repeatedly with 0.5 mL of media. Cell suspensions were filtered through a nylon mesh and fixed in 4% paraformaldehyde for 15 minutes on ice. Cells were washed twice with PBS buffer containing 10% culture grade normal bovine serum, resuspended and counted using a ViCell cell counter. Cells were resuspended at 1×10⁶ cells/mL. Cytospin slides were prepared with 0.5 mL per slide. The slides were air-dried and stored at 80° C. until use.

After blocking, cells were stained with anti-CD41 (rat anti-mouse CD41; clone: MWReg30, cat #133910, Biolegend; diluted 1:150 in PBS contains 10% normal donkey serum) by immunohistochemistry using a BondMax instrument (Leica) and the Rat Polink-2 open kit protocol. Slides were counter stained with hematoxylin, mounted, and cover slipped.

Stained slides were imaged using a Vectra microscope system under 4× and 20× magnification. Images were spectrally unmixed, segmented, then quantified for megakaryocyte count as well as CD41 signal intensity using Inform software. Total, mean and maximum signals as well as signal area was calculated for each category. Data were normalized to total cell numbers and reported as total OD signal or per cell ratio. Data were transferred into Excel and Graph Pad Prism, graphed and analyzed for statistically significant differences.

### C. Results

The results of the IVIg, S1-IVIg, S2-IVIg, and Des-IVIg comparison study are shown in Table 7.

TABLE 7
Total, reticulated, and non-reticulated platelet counts (10⁹/L) on Day 5
and Megakaryocyte count in bone marrow cells (MK/10⁶ BM cells) on Day 4

| | Disease Induction | | | | | |
|---|---|---|---|---|---|---|
| | Isotype | | | | | |
| | Control | | | | | |
| | (1.5 μg) | Anti-CD41 Ab (1.5 μg) | | | | |
| | Treatment | | | | | |
| | | | IVIg | S1-IVIg | S2-IVIg | Desialylated IVIg |
| | | | Dose | | | |
| | Saline | Saline | 1 g/kg | 1 g/kg | 1 g/kg | 1 g/kg |
| n per group | 6 | 6 | 6 | 6 | 6 | 6 |
| Total Platelets | 739 ± 84 | 277 ± 203 | 290 ± 113 | 563 ± 138 | 578 ± 209 | 220 ± 97 |
| [mean ± SD] | | | | | | |
| Reticulated | 61 ± 11 | 98 ± 57 | 131 ± 60 | 184 ± 34 | 261 ± 49 | 130 ± 46 |
| Platelets [mean ± | | | | | | |
| SD] | | | | | | |
| Non-Reticulated | 679 ± 75 | 179 ± 168 | 159 ± 60 | 379 ± 133 | 318 ± 177 | 90 ± 56 |
| Platelets [mean ± | | | | | | |
| SD] | | | | | | |
| Megakaryocytes | 328 ± 132 | 323 ± 51 | 341 ± 162 | 360 ± 31 | 496 ± 115 | 372 ± 87 |
| in Bone Marrow | | | | | | |
| [mean ± SD] | | | | | | |

### Example 4—Comparison of IVIg, rFc, S1-rFc, S2-rFc, and Des-IVIg in a Chronic ITP Mouse Model

The effect of IVIg, rFc, S1-rFc, S2-rFc, and Des-IVIg in an anti-CD41 antibody mediated ITP mouse model was analyzed.

### A. Study Design

Sixty-six to seventy two mice were given 1.5 μg/mouse of rat anti-CD41 antibody (Ab) clone MWReg30 (BioLegend cat #133910) once daily for 4 days (on Days 1, 2, 3 and 4), intraperitoneally. Six to twelve mice were dosed in the same manner with a rat IgG1, k isotype control (BioLegend cat #400414). All mice were dosed once intravenously with saline control, IVIg, recombinant Fc (rFc), S1-rFc, S2-rFc, or Des-IVIg at different doses 1 to 2 hours after the third anti-CD41 Ab injection (Table 8). Mice were bled on Day 4 (4 h after the forth anti-CD41 injection) and on Day 5 (24 h after the forth anti-CD41 Ab injection) to quantitate total platelet and reticulated platelet levels. To confirm that platelet depletion was successful, a subgroup of mice was bled on Day 3, prior to treatment. On Day 5 bone marrow cells were isolated to quantitate megakaryocytes.

TABLE 8
IVIg, rFc, S1-rFc, S2-rFc, and Des-IVIg comparison study details

| | | Induction (1.5 μg IP) | | | | | |
|---|---|---|---|---|---|---|---|
| Group # | n | 4 daily doses | Treatment Agent (200 uL IV) | Dose | | Timing of Dosing | Blood Sampling |
| 1 | 12 | anti-CD41 | Saline | 200 | μL | 1-2 h post 3. anti-CD41 dose | Day 3, Day 4, and Day 5 |
| 2 | 12 | Rat IgG1 | Saline | 200 | μL | 1-2 h post 3. anti-CD41 dose | Day 4 and Day 5 |
| 3 | 12 | anti-CD41 | IVIg Gammagard | 1 | g/kg | 1-2 h post 3. anti-CD41 dose | Day 4 and Day 5 |
| 4 | 12 | anti-CD41 | rFc | 0.3 | g/kg | 1-2 h post 3. anti-CD41 dose | Day 4 and Day 5 |
| 5 | 12 | anti-CD41 | S1-rFc | 0.3 | g/kg | 1-2 h post 3. anti-CD41 dose | Day 4 and Day 5 |
| 6 | 12 | anti-CD41 | S2-rFc | 0.3 | g/kg | 1-2 h post 3. anti-CD41 dose | Day 4 and Day 5 |
| 7 | 12 | anti-CD41 | Des-IVIg | 1 | g/kg | 1-2 h post 3. anti-CD41 dose | Day 4 and Day 5 |

### B. Methods

### ITP Induction in Mice:

In vivo studies were conducted using female C57BL/6 mice (18-22 g, Charles Rivers Labs, MA). All procedures were performed in compliance with the Animal Welfare Act and with the Guide for the Care and Use of Laboratory Animals.

### Quantitation of Total Platelets:

Blood samples were collected by submandibular bleed into EDTA coated tubes, and then run on a VetScan Instrument for platelet level determination. Total platelet levels were analyzed using One-Way ANOVA with Dunnett's or Bonferroni's post-test.

### Quantitation of Reticulated Platelets:

To evaluate and quantitate for the presence of reticulated (young) platelets which contain residual RNA, whole blood was sequentially stained for total platelets (anti-CD61) followed by staining for the RNA with thiazole orange (RNA-binding dye, commercially available as ReticCount Reagent from BD Biosciences). This analysis was performed for blood samples collected on Day 5.

Ten microliters of whole blood was transferred into the bottom of α 5 mL FACS tube. Five microliters of anti-mouse CD61-PE antibody (BD Biosciences) was added directly to the whole blood and samples were mixed thoroughly by pipetting. Samples were incubated at room temperature for 5 minutes in the dark. Two milliliters of ReticCount reagent (BD Biosciences) was added to each sample and samples incubated for a minimum of 30 minutes at room temperature.

Samples were acquired on a FACS Canto flow cytometer (BD Biosciences). Total platelets were identified by forward and side scatter characteristics of the cells and distinguished from erythrocytes by gating on CD61-PE positive events. A total 10,000 platelet events were recorded for each sample. Using FlowJo software, a gate was set on the reticulated platelets (CD61 positive and thiazole orange positive) using samples from isotype control treated mice to achieve a rate of 6-10% reticulated platelets (normal rate). The same gate was then applied to all subsequent samples and treatment groups to calculate percentages of reticulated and non-reticulated platelets. Total counts of reticulated and non-reticulated platelets for each sample were calculated by multiplying the total number of platelets measured in the VetScan Instrument by the percentage of the platelet fraction. Total reticulated and non-reticulated platelet levels were analyzed using One-Way ANOVA with Dunnett's or Bonferroni's post-test.

In addition to the overall platelet quantification using the VetScan Instrument, numbers of reticulated platelets were also determined using ReticCount, anti-CD61-PE labeled Ab and flow cytometry.

### Quantitation of Megakaryocytes in the Bone Marrow:

On Day 5 of the study (ITP-010; 2 days after IVIg treatment and 24 h after the 4^{th} anti-CD41 antibody injection), bone marrow was extracted from one femur per mouse by using a syringe with α 25 gauge needle, flushing the bone shaft repeatedly with 0.5 mL of media. Cell suspensions were filtered through a nylon mesh and fixed in 4% paraformaldehyde for 15 minutes on ice. Cells were washed twice with PBS buffer containing 10% culture grade normal bovine serum, resuspended and counted using a ViCell cell counter. Cells were resuspended at 1×10⁶ cells/mL. Cytospin slides were prepared with 0.5 mL per slide. The slides were air-dried and stored at 80° C. until use.

After blocking, cells were stained with anti-CD41 (rat anti-mouse CD41; clone: MWReg30, cat #133910, Biolegend; diluted 1:150 in PBS contains 10% normal donkey serum) by immunohistochemistry using a BondMax instrument (Leica) and the Rat Polink-2 open kit protocol. Slides were counter stained with hematoxylin, mounted, and cover slipped.

Stained slides were imaged using a Vectra microscope system under 4× and 20× magnification. Images were spectrally unmixed, segmented, then quantified for megakaryocyte count as well as CD41 signal intensity using Inform software. Total, mean and maximum signals as well as signal area was calculated for each category. Data were normalized to total cell numbers and reported as total OD signal or per cell ratio. Data were transferred into Excel and Graph Pad Prism, graphed and analyzed for statistically significant differences.

### C. Results

The results of IVIg, rFc, S1-rFc, S2-rFc, and Des-IVIg comparison study are shown in Table 9.

TABLE 9
Total, reticulated, and non-reticulated platelet counts (10⁹/L) and
Megakaryocyte count in bone marrow cells (MK/10⁶ BM cells) on Day 5

| | Disease Induction | | | | | | |
|---|---|---|---|---|---|---|---|
| | Isotype | | | | | | |
| | Control | | | | | | |
| | (1.5 μg) | Anti-CD41 Ab (1.5 μg) | | | | | |
| | Treatment | | | | | | |
| | | | IVIg | rFc | S1-rFc | S2-rFc | Des IVIg |
| | | | Dose | | | | |
| | Saline | Saline | 1 g/kg | 0.3 g/kg | 0.3 g/kg | 0.3 g/kg | 1 g/kg |
| n per group | 6 | 6 | 6 | 6 | 6 | 6 | 6 |
| Total Platelets | 734 ± 124 | 189 ± 83 | 401 ± 103 | 277 ± 226 | 379 ± 198 | 369 ± 89 | 160 ± 67 |
| [mean ± SD] | | | | | | | |
| Reticulated | 64 ± 10 | 73 ± 39 | 125 ± 42 | 116 ± 55 | 178 ± 60 | 234 ± 95 | 122 ± 56 |
| platelets [mean ± | | | | | | | |
| SD] | | | | | | | |
| Non-Reticulated | 670 ± 117 | 116 ± 70 | 277 ± 106 | 161 ± 191 | 201 ± 157 | 135 ± 46 | 38 ± 25 |
| platelets [mean ± | | | | | | | |
| SD] | | | | | | | |
| Megakaryocytes in | 126 ± 64 | 1.0 ± 0.7 | 275 ± 87 | 237 ± 57 | 218 ± 68 | 345 ± 44 | 81 ± 53 |
| Bone Marrow | | | | | | | |
| [mean ± SD] | | | | | | | |

While the methods have been described in conjunction with various instances and examples, it is not intended that the methods be limited to such instances or examples. On the contrary, the methods encompass various alternatives, modifications, and equivalents, as will be appreciated by those of skill in the art.

What is claimed is:

1. A pharmaceutical preparation comprising sialylated IVIG, wherein at least 65% of branched glycans are sialylated on both the on both the α 1,3 arm and the α 1,6 arm by way of NeuAc-α 2,6-Gal terminal linkages; at least 60% of the branched glycans on the Fab domain are sialylated on both the on both the α 1,3 arm and the α 1,6 arm by way of NeuAc-α 2,6-Gal terminal linkages; and at least 80% of the branched glycan on the Fc domain are sialylated on both the on both the α 1,3 arm and the α 1,6 arm by way of NeuAc-α 2,6-Gal terminal linkages.


SEQUENCE LISTING (3 sequences)

SEQ ID NO: 1 - protein, 418 aa, Homo sapiens
MTRLTVLALLAGLLASSRAGSSPLLAMEWSHPQFEKLEGGGSGGGSGGSWSHPQFEKHAHAHSRKDHLIHNVHKEEHAHAHNKELGTAVFQGPMRRAIRGRSFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHCPG

SEQ ID NO: 2 - protein, 375 aa, Homo sapiens
GSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 3 - protein, 402 aa, Homo sapiens
MIHTNLKKKFSYFILAFLLFALICVWKKGSYEALKLQAKEFQVTKSLEKLAIGSGSQSTSASIKQDSKPGSQVLSHLRVTAKVKPQSPYQVWDKNSSSKNLNPRLQKILKNYLSMNKYKVSYKGPGPGVKFSVEALRCHLRDRVNVSMIEATDFPFNTTEWEGYLPKENFRTKAGPWHRCAVVSSAGSLKSSHLGKEIDSHDAVLRFNGAPVADFQQDVGMKTTIRLMNSQLITTEKQFLKDSLYNEGILIVWDPSLYHADIPNWYKKPDYNFFETYKSYRKLYPSQPFYILRPQMPWELWDIIQEIAPDRIQPNPPSSGMLGIIIMMTLCDQVDVYEFLPSKRKTDVCYYHQKFFDSACTMGAYHPLLFEKNMVKQLNEGTDEDIYIFGKATLSGFRTIHC